(19) United States
(12) Patent Application Publication
(10) Pub. No.: US 2022/0339804 A1
(43) Pub. Date: Oct. 27, 2022

(54) PRODUCTION DEVICE, IN PARTICULAR FOR THE PHARMACEUTICAL INDUSTRY

(71) Applicant: Bausch + Ströbel Maschinenfabrik Ilshofen GmbH + Co. KG, Ilshofen (DE)

(72) Inventor: Markus ZIMMERMANN, Rot am See (DE)

(73) Assignee: Bausch + Ströbel Maschinenfabrik Ilshofen GmbH + Co. KG, Ilshofen (DE)

(21) Appl. No.: 17/763,332

(22) PCT Filed: Sep. 24, 2020
(86) PCT No.: PCT/EP2020/076652

§ 371 Date: Mar. 24, 2022

(30) Foreign Application Priority Data
Sep. 27, 2019 (DE) 10 2019 214 849.2

(51) Int. Cl. B25J 21/00; C12M 1/22; B01L 1/02; F24F 3/16; B25J 9/16

(52) U.S. Cl. CPC B25J 21/00; C12M 23/10; B01L 1/025; F24F 3/16; B25J 9/1689; B01L 2300/0681; B01L 2200/141

(57) ABSTRACT

A production device including an isolator housing to receive functional components of the production device and products in an interior of the production device and to hermetically seal the components from the surroundings. The production device includes at least one robot received in the isolator housing, wherein the robot is one or more of operable to carry out specific tasks or operable to be remotely controllable by means of a remote controller. The production device includes at least one linear movement unit received in the isolator housing and configured to move the at least one robot long a linear movement axis. The production device includes at least one control unit to control one or more of a movement or an operation of the at least one robot.

The present invention relates to a production device, in particular for the pharmaceutical industry.

Because they handle dangerous or sensitive substances, such production devices, with which a wide variety of processing operations can be carried out on corresponding products, usually have a barrier between their surroundings and, for example, human operators and the products to be processed. Such barriers are typically designed as isolator housings having glove openings that said operators can use to perform various activities within the isolator housing.

In the case of processing aseptic products, external interventions, i.e. interventions undertaken by the human operators, among others, represent the greatest hazard for contamination during aseptic production. On the other hand, when processing toxic products, a barrier must always be provided between human operators and the products, wherein as a rule a negative pressure is also provided in the isolator housing having glove openings, so that even in the event of a leak, the escape of potentially toxic substances can be minimised or prevented entirely. In any case, adequate measures must be taken to protect the human operator from toxic substances when said substances are being processed.

The complexity of modern production devices, particularly in the pharmaceutical industry, such as filling machines, for example, means that there are numerous possible sources of error at many locations in such production devices. On the other hand, however, recurring work steps, such as changing Petri dishes (microbe collectors) or so-called “aseptic assembly” processes at the start of production, have not yet made it possible to forego providing glove openings for operator intervention, since for successful production interventions must be performed regularly in the interior of the isolator housing in order to carry out routine or unscheduled actions there.

It is therefore the object of the present invention to provide an improved such production device in which, on the one hand, the greatest source of contamination for the products disposed in the isolator housing is eliminated, and, on the other hand, the greatest hazard for human operators outside the isolator housing is eliminated, and which production device nevertheless permits at least equally reliable operation compared to the prior art devices discussed above.

To attain this object, the inventive production device comprises an isolator housing which is designed to receive functional components of the production device and products in the interior of said production device and to hermetically seal said components and products from the surroundings, at least one robot which is received in the isolator housing and can be operated so as to carry out specific tasks and/or so as to be remotely controllable by means of a remote controller, at least one linear movement unit which is received in the isolator housing and is designed to move the at least one robot along a linear movement axis, and at least one control unit which is designed to control the movement and/or the operation of the at least one robot.

By thus inventively providing a linearly movable robot within the isolator housing, the above-described glove openings into the isolator housing can be completely eliminated, so that not only is it possible to improve the hermetic sealing of the interior of the isolator housing from its surroundings and thus prevent contamination, but it is also possible to see into the isolator housing better during the operation of the production device, since gloves no longer obscure anything.

Furthermore, the structural integrity of the isolator housing is also improved, since it is possible to eliminate the weakening of the housing caused by providing rings for the glove openings, and this also applies to protective covers or laser scanners to be provided there.

According to the invention, a single control unit can be provided in the production device, which controls both the movement and the operation of the at least one robot in a coordinated manner. Alternatively, separate control units which are operatively coupled to one another could also be provided for the linear movement unit and the robot. In one further embodiment, the control unit could be formed by a control device of a higher-level module for controlling a more complex overall process, which module, in addition to its other tasks, also controls the movement and operation of the robot, improving the integration of these components into the overall production operation. In any case, the control unit can comprise devices known per se, such as, for example, microprocessors or microcontrollers and associated memory units, as well as suitable communication means.

In one embodiment, the inventive production device can furthermore comprise a circulation device designed to cause air to circulate within the isolator housing, wherein the circulation device can preferably comprise a filter unit. By doing without glove openings, improved circulation can be achieved within the isolator housing, and double discs, for example, can be used in the circulation device.

Alternatively or in addition, the inventive production device can comprise a pressure regulating device designed to adjust the internal pressure within the isolator housing with respect to the ambient pressure. Depending on the nature of the production device and, in particular, the products to be processed therein, an internal pressure that is higher or lower than the ambient pressure can be desirable and can be set, either to prevent contamination from entering the isolator housing or to prevent, or at least reduce, toxic substances from escaping from the isolator housing.

Furthermore, in one particularly simple embodiment, the linear movement unit, or at least one of the linear movement units, can comprise a rail extending along the movement axis. The linear movement of the at least one robot can be driven and actuated on this rail by means of suitable slides or the like. Alternatively, however, other embodiments of the at least one linear movement unit are also possible, for example linearly telescopic mountings to which the corresponding robot is attached, or the like.

Since the inventive production device can be provided and designed for a wide variety of purposes and processing operations, the at least one robot can also be provided and designed for a wide variety of activities.

For example, at least one robot can be considered which is designed to change Petri dishes and/or to install filling needles and/or to carry out cleaning processes and/or to pick up and set down products and/or to diagnose errors and/or to correct said errors. It is understood that this list is not exhaustive, but rather that the advantageous effects of the present invention do not relate to the specific intended activity of the specific robot or robots, but can be set universally without the type or the intended activity of the robot playing a role, although it may of course be necessary to adapt further components of the device to the characteristics of the robot, for example adjusting the linear movement unit to the shape and weight of the robot and/or adjusting the control unit to the desired functional scope of the robot.

Furthermore, the at least one robot can comprise a camera and/or at least one sen-sor unit, wherein camera data and/or sensor data output therefrom are output to a display and/or control device and/or can be used for autonomous activities of the robot. In particular in the event that remote control of the robot is provided, the camera data can be displayed directly on a display device for an operator of the remote control, while of course said data can also be used for automatic image recognition and processing, possibly together with or as an alternative to sensor data from the at least one sensor for regulating processes of the robot.

Furthermore, the inventive production device can comprise a transport device for products to be processed in the isolator housing, which transport device can preferably be arranged above the linear movement unit or one of the linear movement units. This transport device can make it possible for the products to be processed to be transported through the interior of the isolator housing or can also bring about more complicated movements of the products to be processed, for example lock functions or the like, during which processes the corresponding products can be supplied to different transport routes.

Although, as mentioned, the advantageous effect of the inventive production device is not directly related to the type of processing it actually carries out, in one embodiment the functional components can comprise a filling machine for filling products. In the pharmaceutical industry in particular, hermetic sealing of the interior of the isolator housing from its surroundings is often necessary due to the processing of aseptic or toxic products.

Furthermore, the inventive production device can be embodied such that the isolator housing comprises an access lock through which products and/or tools can be introduced into the interior. It is understood that the term “access lock” is to be construed broadly and that such a lock can also be used to unload products and/or tools. Accordingly, for example, two access locks of this type can be provided on the isolator housing of the inventive production device, wherein during normal operation of the device one of said two access locks is used to load products and/or tools in, while the other is used to unload them. In connection with the above-mentioned lock function of a transport device for products within the isolator housing, however, embodiments having more than two access locks are certainly also possible. Of course, the inventive production device can also be integrated into higher-level process units or complex production chains, so that the products can be advanced to the production device from upstream machines and/or the products processed by the production device can be forwarded to downstream machines. Because these processes are carried out automatically using suitable means, it is possible to achieve excellent integration of the inventive device into higher-level processes and systems.

Further advantages and features of the present invention will become even more clear from the following description of embodiments thereof when the invention is considered together with the accompanying drawings. In detail, in the drawings:

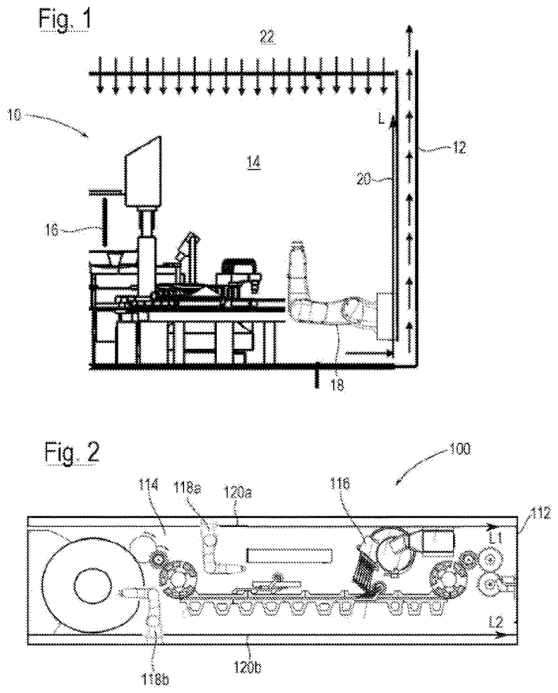

FIG. 1 is a schematic view of a first embodiment of an inventive production device; and

FIG. 2 a schematic view of a second embodiment of an inventive production device.

FIG. 1 initially shows a schematic view of an inventive production device which is designated quite generally with the reference numeral 10. The production device 10 comprises an isolator housing 12 which has an interior 14 and hermetically seals the latter against the surroundings of the production device 10, wherein one or more access locks (not shown in FIG. 1), through which products and/or tools can be introduced into the interior 14, can be provided.

Received in the interior 14 of the isolator housing 12 are functional components of the production device 10, in this case a schematically illustrated filling machine 16 for filling containers in the pharmaceutical industry, wherein in this case the containers correspond to the products to be processed within the meaning of the present application.

Furthermore, a robot 18, which can be moved along a linear movement axis L by means of a linear movement unit 20, is disposed within the interior 14 of the isolator housing 12. Here, in the view from FIG. 1, the linear movement axis L runs vertically, so that the robot 18 can also be moved vertically. Although the robot 18 in FIG. 1 is shown as a typical 6-axis robot, as it is often used in the processing industry, other types of robots could also be used in the device 10 from FIG. 1, of course.

Finally, a series of arrows in FIG. 1 indicates air circulation that can be caused, for example, by a circulation device provided in plenum 22 (not shown in FIG. 1), wherein the circulation device can further optionally comprise at least one filter unit.

In the embodiment shown in FIG. 1, the linear movement unit 20 is embodied in the form of a rail system on which the robot 18 can be moved, so that the linear movement axis L acts as the seventh axis, so to speak, of the 6-axis robot. The robot 18 can therefore take on different tasks within the interior 14 of the isolator housing 12, which tasks will be discussed further below in connection with different functional components, wherein in particular in FIG. 1 and in the context of the filling machine 16 shown there it is possible for the robot 18 to introduce containers to be filled into the filling machine 16 and remove said containers therefrom, wherein the containers are first loaded into the interior 14 of the isolator housing 12 through the above-mentioned access lock (not shown).

A second embodiment of an inventive production device is now shown in FIG. 2 and denoted with the reference symbol 100, wherein components similar to those from FIG. 1 are denoted by the same reference symbols, but increased by 100. In particular, the production device 100 from FIG. 2 also comprises an isolator housing 112 having an interior 114 in which a filling machine 116 is provided, said filling machine itself acting as a functional component of the production device 100.

In contrast to the production device 10 from FIG. 1, however, the production device 100 from FIG. 2 comprises two robots 118a and 118b which are designed analogously to the robot 18 from FIG. 1 but are assigned to two different linear movement units 120a and 120b, the linear movement axes L1 and L2 of which also run horizontally and not vertically. The two robots 118a and 118b can thus be operated independently of one another or in a manner coordinated with one another and can be moved linearly along the two axes L1 and L2.

Both in the embodiment from FIG. 1 and in the embodiment from FIG. 2, at least the robots 18 or 118a and 118b are assigned a control unit (not shown here) which can control the operation of the corresponding robot, on the one hand, and also possibly its movement by the linear movement unit, on the other hand, wherein this linear movement unit could alternatively also be assigned its own control unit which could accordingly be operatively coupled to the control unit of the corresponding robot.

Furthermore, it is possible to couple the control units of the robots and/or linear movement units to the control units for higher-level processes, such as, e.g., to couple the operation of the corresponding functional components, i.e. the filling machines 18 or 118, or to provide a higher-level control unit for all of the components just mentioned in order to be able to assure coordinated and smooth operation of all of the aforesaid components.

It has been shown that providing the robots 18 and 118a, 118b and the associated linear movement units 20 or 120a, 120b makes it possible to do without providing glove openings in the corresponding isolator housings 12 and 112, so that the advantages of the present invention discussed above are achieved. In the following, a few examples are cited of tasks that can be carried out in inventive devices using the interaction of provided robots with the linear movement units assigned to them.

In one arrangement for an “aseptic assembly,” an access lock can be opened automatically/manually from the outside and the corresponding needles with hose can be introduced as the product to be processed using a transfer container or a beta bag. The corresponding robot can pick up the needles with hose and install them in a needle holder. An associated peristaltic pump or a time/pressure metering device can be attached outside the isolator housing. In very general terms, it is understood that essentially any filling systems can be used in variants of this embodiment in the context of the inventive device and that any division between components arranged inside and outside the isolator housing can be provided within the framework of general considerations when optimising the device.

For automated changing of Petri dishes as microbe collectors, an access lock can also be opened automatically or manually from the outside and the Petri dishes can in turn be introduced using a transfer container or beta bag. The provided robot can then pick them up and install them in a positioned manner in a Petri dish silo. During operation, the robot thus moves along its linear movement axis to different positions and changes the Petri dishes, which change in some embodiments can be controlled manually or, for example, after a certain time as part of a preset program. In this context, since the robot is moved only very slowly, it is possible to prevent air within the isolator housing from being swirled too much, which could distort the results of the sequence to be carried out.

Another use for the linearly movable robot could be the manually controlled or automatic detection and, if necessary, elimination, of faults in components within the isolator housing; for example, using a camera attached to it, the robot could detect individual components within the isolator housing and could be moved using a manual remote controller into a suitable position for recording further detailed camera images or for manipulating, for example repairing, the specific component directly using suitable tools. In one alternative embodiment, the corresponding robot could also act proactively or independently and, if necessary, carry out the described steps automatically without remote control, for example based on the principles of machine learning.

Such a robot could also be used for washing or disinfection processes in that it picks up washing guns disposed in the isolator housing and runs a programmed cycle in which, for example, water or CO₂ snow is sprayed onto components to be cleaned within the isolator housing. In this case, again, it is possible for an operator to use a remote controller for manual control, or for a specific cleaning program to be run automatically. Furthermore, such cleaning can be additionally supported as required by permanently installed washing nozzles and the positions of machine stops can be kept in the control unit of the robot and the cleaning time can be extended at specific points. It could also be possible to have the corresponding robot dismantle individual components within the isolator housing in order to enable more efficient cleaning thereof. Furthermore, after the cleaning process has been completed, the robot can also perform a drying operation, for example in that the robot picks up a sterile blow-out gun and uses it to dry the cleaned components.

Finally, the robot could also be used in automatic weighing processes in that the robot picks up products within the isolator housing and places them on a load cell, while in a similar manner it can also pick up products for washing processes and position them over a washing device.

1. A production device comprising:
an isolator housing to receive functional components of the production device and products in an interior of the production device and to hermetically seal the components from the surroundings;
at least one robot received in the isolator housing, wherein the robot is operable to carry out specific tasks;
at least one linear movement unit received in the isolator housing and configured to move the at least one robot along a linear movement axis; and
at least one control unit to control one or more of a movement or an operation of the at least one robot.

2. The production device of claim 1, further comprising a circulation device designed to cause air to circulate within the isolator housing.

3. The production device of claim 1, further comprising a pressure regulating device designed to adjust an internal pressure within the isolator housing with respect to an ambient pressure.

4. The production device of claim 1, wherein at least one of the at least one linear movement unit comprises at least one rail extending along the linear movement axis.

5. The production device of claim 1, wherein the at least one robot is configured to perform one or more of (a) changing Petri dishes, (b) installing filling needles, (c) carrying out cleaning processes, (d) picking up and setting down products, (d) diagnose diagnosing errors, or (e) correcting the errors.

6. The production device of claim 1, wherein the at least one robot comprises one or more of a camera or at least one sensor unit, wherein data generated by at least one of the camera or the sensor unit data is output to one or more of a display device or a control device .

7. The production device of claim 1, further comprising a transport device configured to transport products to be processed in the isolator housing.

8. The production device of claim 1, wherein the functional components include a filling machine for filling products.

9. The production device of claim 1, wherein the isolator housing comprises an access lock through which products can be introduced into the interior of the production device.

10. The production device of claim 2, wherein the circulation device comprises a filter unit.

11. The production device of claim 7, wherein the transport device is arranged above the at least one linear movement unit.

12. The production device of claim 1, wherein the isolator housing comprises an access lock through which tools can be introduced into the interior of the production device.

13. The production device of claim 1, wherein the at least one robot comprises one or more of a camera or a sensor unit, wherein data generated by at least one of the camera or the sensor unit is used for autonomous activities of the robot.

14. The production device of claim 1, wherein the robot is remotely controllable by means of a remote controller;
